(19) United States
(12) Patent Application Publication
(10) Pub. No.: US 2026/0207865 A1
(43) Pub. Date: Jul. 23, 2026

(54) INHALATION ARRANGEMENT

(71) Applicant: URSATEC GmbH, Tholey (DE)

(72) Inventors: Lars THELIN, Falun (SE); Fredrik BOKVIST, Svärdsjö (SE)

(73) Assignee: URSATEC GmbH, Tholey (DE)

(21) Appl. No.: 19/135,973

(22) PCT Filed: Dec. 5, 2023
(86) PCT No.: PCT/SE2023/051222

§ 371 Date: Jun. 5, 2025

(30) Foreign Application Priority Data
Dec. 6, 2022 (SE) 2251429-3

(51) Int. Cl. A61M 15/00; A61M 11/00; A61M 15/08

(52) U.S. Cl. CPC A61M 15/0021; A61M 11/003; A61M 11/006; A61M 15/0013; A61M 15/08; A61M 2202/0007; A61M 2202/02; A61M 2209/088; A61M 2210/0618; A61M 2210/0625

(57) ABSTRACT

Inhalation arrangement comprising a pacifier part and a nose part. The pacifier part comprises a nipple configured for being provided inside the mouth of a user of the inhalation arrangement, said nipple comprising a passageway through which passageway a fluid can pass and a housing connected to the nipple. The housing comprises a pacifier part drug compartment which is fluidly connected to the passageway of the nipple, a pacifier part drug inlet configured for being connected to an external drug providing device, a gas inlet and a nose part connector configured to be connected to a pacifier part connector of the nose part. The nose part comprises a nose part drug compartment, a nose attachment part configured for transferring drug from the nose part drug compartment into a nose of a user of the inhalation arrangement and the pacifier part connector which is configured to be connected to the nose part connector of the pacifier part and hereby fluidly connecting the nose part drug compartment and the pacifier part drug compartment.

### TECHNICAL FIELD

The present invention relates to an inhalation arrangement.

### BACKGROUND

Inhalation of drugs may be complicated especially for small children. Coordination of breath and drug inhalation is problematic and mouthpieces or masks may be hard to use for small children. For very small children inhalation drug is sometimes delivered via a nasal cannula. However, the dose of drug actually inhaled by the child is difficult to control because a part of the drug will stay in the nose and another part of the drug that is pushed through the nasal cannula will escape out to the surroundings because the breathing of the child is not coordinated with the drug delivery. Small children are neither inhaling deep nor regularly and also have a very short breathing cycle. Hereby a delivered dose of inhalation drug is hard to determine. Current understanding is that approximately 1-10% of the drug reaches the target organ, ie the lungs, with standard devices.

### SUMMARY

An object of the present invention is to provide an improved inhalation arrangement for small children.

This is achieved by an inhalation arrangement according to claim 1.

According to the invention an inhalation arrangement is provided, wherein said inhalation arrangement comprises a pacifier part and a nose part which are configured to be connected to each other, said pacifier part comprising:
a nipple configured for being provided inside the mouth of a user of the inhalation arrangement, said nipple comprising a passageway, which is passing through the nipple, through which passageway a fluid can pass; and
a housing connected to the nipple, said housing comprising:
   a pacifier part drug compartment which is fluidly connected to the passageway of the nipple;
   a pacifier part drug inlet configured for being connected to an external drug providing device, said pacifier part drug inlet being connected to the pacifier part drug compartment and configured for transferring a drug provided from a connected external drug providing device into the pacifier part drug compartment;
   a gas inlet allowing air or other gas to enter into the pacifier part drug compartment; and
   a nose part connector configured to be connected to a pacifier part connector of the nose part,
      said nose part comprising:
a nose part drug compartment;
a nose attachment part connected to the nose part drug compartment and configured for transferring drug from the nose part drug compartment into a nose of a user of the inhalation arrangement; and
the pacifier part connector which is configured to be connected to the nose part connector of the pacifier part and hereby fluidly connecting the nose part drug compartment and the pacifier part drug compartment.

Hereby an inhalation arrangement is achieved by which the child can inhale both via the mouth and the nose. Drug can be inhaled via the mouth through the pacifier part of the inhalation arrangement and via the nose through the nose part of the inhalation arrangement. Hereby drug can be delivered even if the child has obstructed nose. Furthermore, thanks to the pacifier part drug compartment, which is acting as a drug reservoir, the child can inhale the drug at its own pace. Hereby less drug will be lost to the surrounding and upper airway mucosa. Also the dose of drug delivered to the child can be better controlled and monitored. Furthermore, the pacifier part of the inhalation arrangement will probably be better accepted by a small child than other types of mouth pieces or masks for inhalation and furthermore the drug will be delivered further into the mouth of the child thanks to the nipple reaching into the mouth of the child. Hereby a larger part of the drug will reach all the way down to the trachea or lungs and less drug will be stuck in the mouth and nose compared to prior art inhalers. This means that the predictability of given dose is considerably more accurate than in prior art.

In one embodiment of the invention the nose part further comprises a nose part drug inlet configured for being connected to an external drug providing device, said nose part drug inlet being connected to the nose part drug compartment and configured for transferring a drug provided from a connected external drug providing device into the nose part drug compartment. Hereby drug can be supplied both via the nose part and the pacifier part.

In one embodiment of the invention said nose part further comprises at least one gas flow inlet connected to the nose part drug compartment and configured for transferring gas from a connected external gas providing device into the nose part drug compartment. Hereby gas such as air and/or oxygen can be provided in the same way as in a conventional continuous positive airway pressure, CPAP, system, or a high flow nasal canula, HFNC, system.

In one embodiment of the invention said pacifier part drug inlet comprises an aerosolization device through which said drug is transferred into the pacifier part drug compartment. If a nose part drug inlet is provided in the nose part an aerosolization device can be provided also to the nose part drug inlet. Hereby a drug can be supplied into the inhalation arrangement and the drug will be aerosolized when transferred through the aerosolization device.

In one embodiment of the invention the aerosolization device comprises a fine pore membrane, whereby a drug provided from a connected external drug providing device will be aerosolized when transferred through the fine pore membrane. Hereby an aerosolized drug suitable for inhalation can be transferred to a user via the inhalation arrangement according to the invention.

In one embodiment of the invention the pacifier part drug inlet is configured for connection to a source of aerosolized drug.

In one embodiment of the invention the nose part connector is a closed valve when no nose part is connected to the pacifier part and is an open valve when a nose part is connected to the pacifier part. Hereby the pacifier part drug compartment and the nose part drug compartment are fluidly connected when the pacifier part and the nose part are connected. However, when the nose part is not connected to the pacifier part the pacifier part can be used on its own.

In one embodiment of the invention the nose attachment part comprises nostril inert parts or a nose cap. Hereby inhalation drug can be efficiently delivered into the nose of a user of the inhalation arrangement.

In one embodiment of the invention the pacifier part further comprises a shield, wherein the nipple is protruding from a first surface of the shield and the housing is provided on a second surface of the shield, which second surface is opposite to the first surface.

In one embodiment of the invention the shield further comprises hooks for allowing securing the inhalation arrangement to a head of a user.

### BRIEF DESCRIPTION OF THE DRAWINGS

FIG. 1 is a perspective view of an inhalation arrangement according to one embodiment of the invention.

FIG. 2 is a perspective view of the inhalation arrangement as shown in FIG. 1 with arrows added illustrating a possible flow of inhalation drug through the inhalation arrangement.

FIG. 3 is a perspective view of an inhalation arrangement according to another embodiment of the invention.

FIG. 4 is a perspective view of an inhalation arrangement according to another embodiment of the invention.

FIG. 5 is a perspective view of an inhalation arrangement according to another embodiment of the invention.

FIG. 6 is a perspective view of an inhalation arrangement according to another embodiment of the invention.

FIG. 7 is a perspective view of a pacifier part of an inhalation arrangement according to one embodiment of the invention.

### DETAILED DESCRIPTION OF EMBODIMENTS

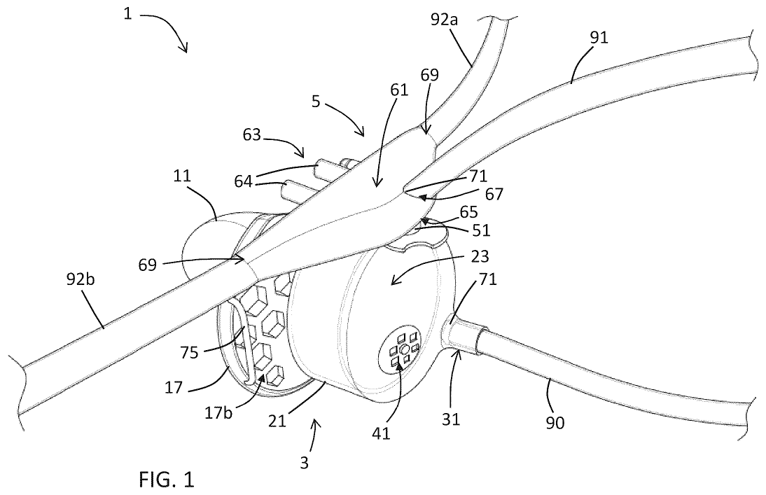

In FIGS. 1-7 a few different embodiments of inhalation arrangements 1; 101; 201; 301; 401 according to the invention are schematically shown. Many details are the same and are therefore given the same or corresponding reference numbers. The different embodiments are described in more details below but first a general description of the invention referring to all the figures is given.

The inhalation arrangement 1; 101; 201; 301; 401 according to the invention is an inhalation arrangement which is especially suitable for small children and comprises a pacifier part 3; 3′ and a nose part 5; 5′; 5″; 5″ which are configured to be connected to each other. The pacifier part 3; 3′ and the nose part 5; 5′; 5″; 5′″ can in some embodiments be fixedly connected to each other and in other embodiments they are releasably connectable to each other. The pacifier part 3; 3′ comprises a nipple 11 configured for being provided inside the mouth of a user of the inhalation arrangement 1; 101; 201; 301; 401. The nipple 11 comprises a passageway 13 through which a fluid can pass. The pacifier part 3; 3′ comprises further suitably a shield 17 connected to the nipple 11 as in a conventional pacifier. The shield 17 is provided for assuring the nipple is not swallowed by the child. The pacifier part 3; 3′ comprises furthermore a housing 21 connected to the shield 17. The housing 21 is provided on an opposite side of the shield 17 compared to the location of the nipple 11, i.e. the nipple 11 and the housing 21 are connected to opposite surfaces of the shield 17. The nipple 11 is protruding from a first surface 17a of the shield 17 and the housing 21 is provided on a second surface 17b of the shield 17, which second surface 17b is opposite to the first surface 17a. The housing 21 comprises a pacifier part drug compartment 23. The pacifier part drug compartment 23 acts as a reservoir for a drug to be inhaled via the inhalation arrangement 1; 101; 201; 301; 401, i.e. a drug to be administered to a child via the inhalation device is first collected in the pacifier part drug compartment 23. Hereby, thanks to the possibility to collect the drug in a reservoir on its way to the user there is a decreased risk that drug is lost, for example by escaping via the user's mouth. Thanks to the pacifier part drug compartment 23 acting as a reservoir for the drug, the drug will instead be administered in coordination with the inhaling by the user. This also allows the child to inhale the drug with tidal breathing and no coordination is needed. Drug is administered to the user from the pacifier part drug compartment 23 when the user inhales via the nipple of the inhalation arrangement. The pacifier part drug compartment 23 is fluidly connected to the passageway 13 of the nipple 11.

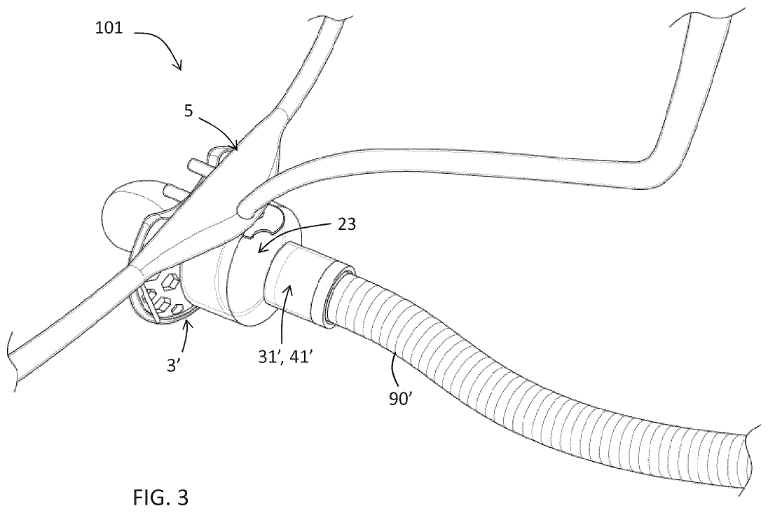

The housing 21 comprises furthermore a pacifier part drug inlet 31 configured for being connected to an external drug providing device. Said pacifier part drug inlet 31 is connected to the pacifier part drug compartment 23 and configured for transferring a drug provided from a connected external drug providing device into the pacifier part drug compartment 23. The housing 21 comprises furthermore a gas inlet 41 connected to the pacifier part drug compartment 23 and allowing air or other gas to enter into the pacifier part drug compartment 23. In some embodiments the pacifier part drug inlet 31 and the gas inlet 41 are combined which will be further described below in relation to FIG. 3.

The gas inlet 41 comprises suitably a one way valve, whereby air or other gas can be drawn into the pacifier part drug compartment 23 by the user when the user inhales but no drug can escape out from the gas inlet 41. The gas inlet 41 is necessary for allowing the user to inhale freely which will greatly improve drug administration through the airflow that is created with inhalation.

The housing 21 comprises furthermore a nose part connector 51 configured to be connected to the nose part 5; 5′; 5″; 5′″. As discussed above the pacifier part 3; 3′ and the nose part 5; 5′; 5″; 5′″ may in some embodiments be permanently connected to each other and in other embodiments they are releasably connectable to each other. Hereby the nose part connector 51 can either be fixedly connected to the nose part 5; 5′; 5″; 5′″ or configured for allowing connection of the nose part 5; 5′; 5″; 5′″ to the pacifier part 3; 3′.

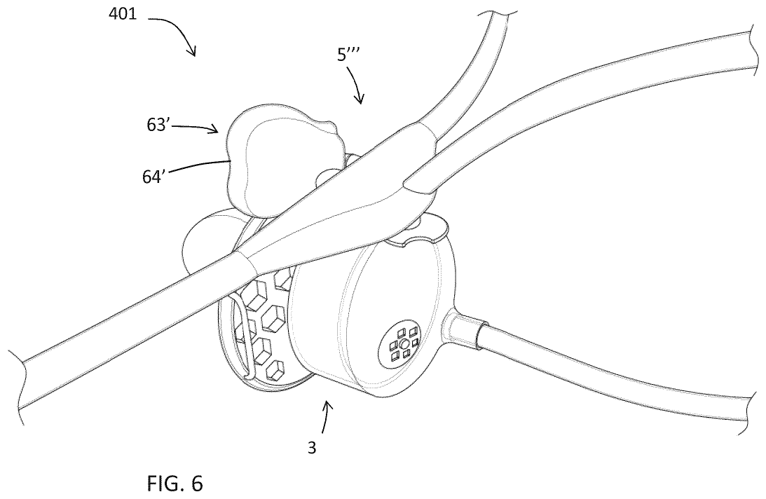

The nose part 5 of the inhalation arrangement 1; 101; 201; 301; 401 comprises a nose part drug compartment 61 in which a drug to be inhaled by a user of the inhalation arrangement 1; 101; 201; 301; 401 can be collected before inhalation. The nose part 5 comprises furthermore a nose attachment part 63; 63′, which for example comprises two nostril insert parts 64 as shown in FIGS. 1-5 or a nose cap 64′ as shown in FIG. 6. The nose attachment part 63; 63′ is connected to the nose part drug compartment 61 and configured for transferring drug from the nose part drug compartment 61 into a nose of a user of the inhalation arrangement 1; 101; 201; 301; 401. The nose part 5; 5′; 5″; 5′″ comprises furthermore a pacifier part connector 65 configured to be connected to the pacifier part 3; 3′. As discussed above the pacifier part connector 65 is in some embodiments fixedly connected to the nose part connector 51 of the pacifier part 3; 3′ and in other embodiments configured for allowing releasable connection to the nose part connector 51 of the pacifier part 3; 3′. When the pacifier part 3; 3′and nose part 5; 5′; 5″; 5′″ are connected, i.e. when the pacifier part connector 65 and the nose part connector 51 are connected, the nose part drug compartment 61 and the pacifier part drug compartment 23 are fluidly connected. Hereby drug which is transferred from an external drug providing device into the pacifier part drug compartment 23 can be transferred into the mouth of a user via the nipple 11 but also into the nose part drug compartment 61 and further via the nose attachment part 63; 63′ into a nose of the user of the inhalation arrangement 1; 101; 201; 301; 401. Hereby drug is efficiently administered both via mouth and nose of the user of the inhalation arrangement. When the pacifier part 3; 3′ and the nose part 5; 5′; 5″; 5′″ are not connected the nose part connector 51 is in some embodiments a closed valve whereby the pacifier part 3; 3′ can be used without the nose part 5; 5′; 5″; 5′″. The valve of the nose part connector 51 will be open when the pacifier part and the nose part are connected.

In some embodiments of the invention the nose part 5; 5′″ further comprises a nose part drug inlet 67 connected to the nose part drug compartment 61 and configured for transferring a drug from a connected external drug providing device into the nose part drug compartment 61.

In some embodiments of the invention the nose part 5; 5″; 5′″ further comprises at least one gas flow inlet 69 connected to the nose part drug compartment 61 and configured for transferring gas from a connected external gas providing device into the nose part drug compartment 61. Hereby air or extra oxygen can be administered together with the drug conveniently.

In some embodiments of the invention the pacifier part drug inlet 31 comprises an aerosolization device 71 through which said drug is transferred into the pacifier part drug compartment 23. The aerosolization device 71 can be a fine pore membrane, also called a nozzle. Such a fine pore membrane will aerosolize the drug when the drug is forced through the fine pore membrane. In other embodiments the pacifier part drug inlet 31 is configured for connection to a source of already aerosolized drug. This is shown in FIG. 3. If the nose part 5; 5′″ comprises a nose part drug inlet 67 (as shown in FIGS. 1-3 and 6) the nose part drug inlet 67 may also comprise an aerosolization device 71, such as a fine pore membrane.

In some embodiment of the invention the shield 17 comprises hooks 75 for allowing securing of the inhalation arrangement 1; 101; 201; 301; 401 to a head of a user.

A few specific embodiments are now described in more detail with reference to respective Figure.

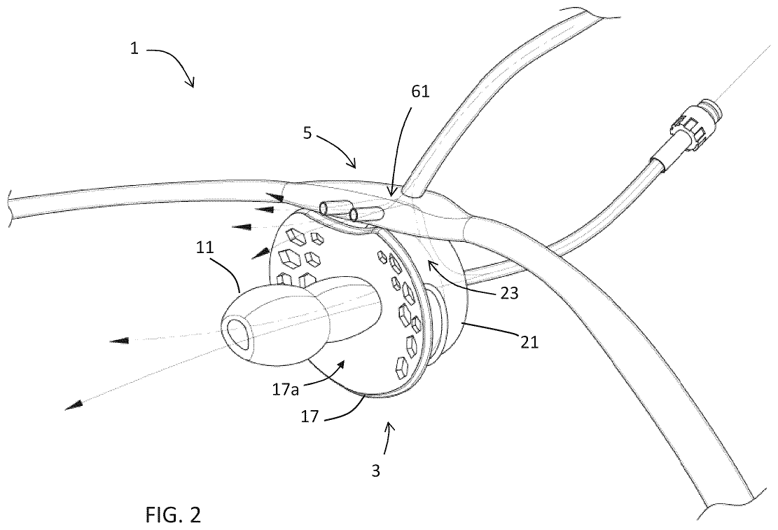

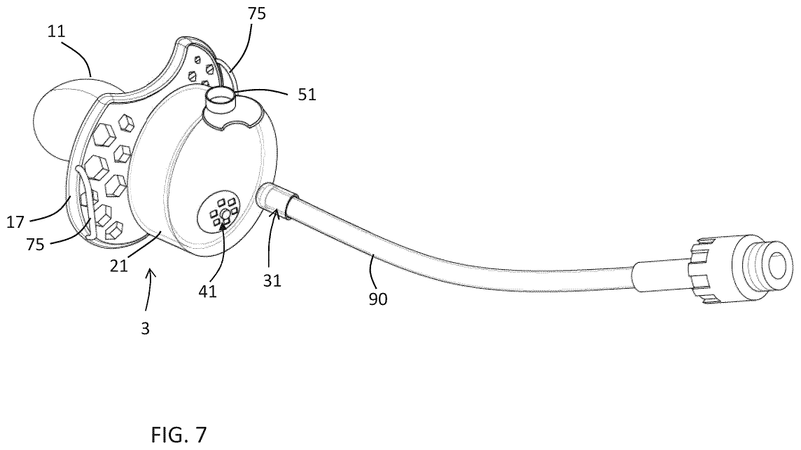

In FIG. 1 an inhalation arrangement 1 according to one embodiment of the invention is shown in a perspective view. In FIG. 2 the same inhalation arrangement 1 is shown in another perspective view and with an added illustration of a flow of drug through the inhalation arrangement 1. The flow of drug is illustrated by arrows which will be described in more details below. The inhalation arrangement 1 comprises a pacifier part 3 and a nose part 5 which here are connected to each other. In some embodiments the nose part 5 can be disconnected and the pacifier part 3 may be used on its own. Possibly also the nose part 5 can be used on its own. A separate pacifier part 3 is shown in FIG. 7. As described for a general embodiment above the pacifier part 3 comprises a nipple 11 and a housing 21. A shield 17 is suitably also provided which could be a part of the housing 21 or a separate part provided in between the nipple 11 and the housing 21. The housing 21 comprises a pacifier part drug compartment 23, a pacifier part drug inlet 31, a gas inlet 41 and a nose part connector 51 as described above. The nose part connector 51 can be better seen in FIG. 6. The nose part connector 51 can be a valve which is closed when the nose part 5 is not connected to the pacifier part 3, as shown in FIG. 7. Hereby the pacifier part 3 can be used on its own, without a connected nose part 5. The pacifier part drug inlet 31 is configured for being connected to an external drug providing device (not shown). A tube 90 is shown connected to the pacifier part drug inlet 31 and said tube 90 will be connected in its other end to an external drug providing device.

The nose part 5 comprises a nose part drug compartment 61, a nose attachment part 63 and a pacifier part connector 65 (hidden in the Figures but provided for mating with the nose part connector 51) as described above. The nose attachment part 63 comprises in this embodiment two nostril insert parts 64. The nose attachment part 63 can instead comprise a nose cap 64′ as shown in FIG. 6, i.e. all embodiments as illustrated in FIGS. 1-5 can comprise a nose cap 64′ instead of nostril insert parts 64 or another device for transferring the drug into the nose of the user.

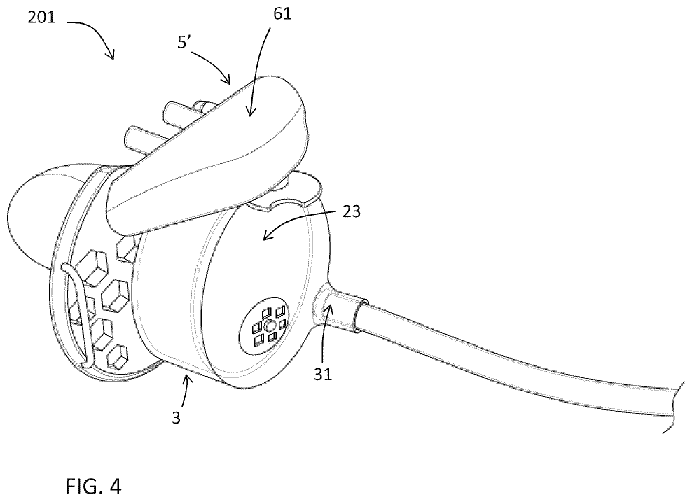

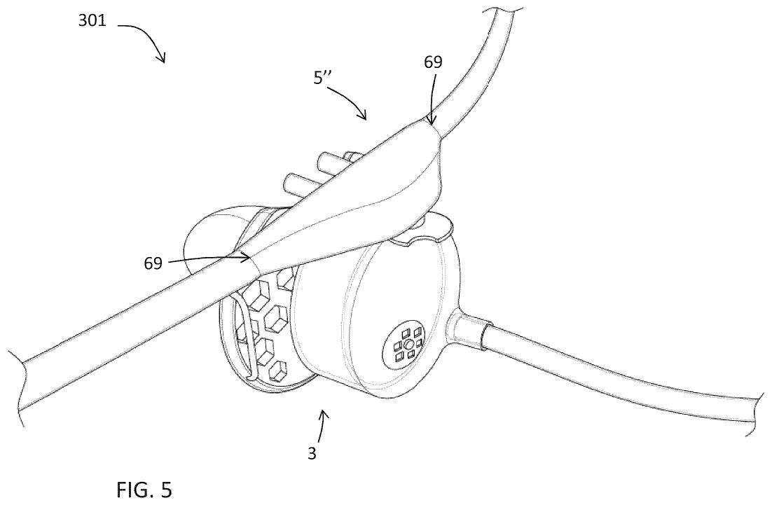

In the embodiment as shown in FIGS. 1 and 2, the nose part 5 further comprises a nose part drug inlet 67 configured for being connected to an external drug providing device (not shown). A tube 91 is shown connected to the nose part drug inlet 67 and said tube 91 will be connected in its other end to an external drug providing device. The nose part drug inlet 67 is connected to the nose part drug compartment 61 and configured for transferring a drug provided from a connected external drug providing device into the nose part drug compartment 61. Such a nose part drug inlet 67 and separate connection of the nose part 5 to a drug providing device is however not necessary, as shown in the embodiments which are illustrated in FIGS. 4 and 5.

In the embodiment of FIGS. 1 and 2 the nose part 5 further comprises two gas flow inlets 69 connected to the nose part drug compartment 61 and configured for transferring gas from a connected external gas providing device into the nose part drug compartment 61. Tubes 92a, 92b are shown connected to the gas flow inlets 69 and said tubes 92a, 92b are connected in their opposite ends to a source of gas, such as air and/or oxygen in the same way as in a conventional continuous positive airway pressure, CPAP, system, or a high flow nasal canula, HFNC.

In this embodiment the pacifier part drug inlet 31 comprises an aerosolization device 71 through which said drug is transferred into the pacifier part drug compartment 23. The aerosolization device 71 comprises a fine pore membrane, whereby a drug provided from a connected external drug providing device will be aerosolized when transferred through the fine pore membrane. Such an aerosolization device 71, for example in the form of a fine pore membrane, may also be provided to the nose part drug inlet 67 of the nose part 5. Hereby drug supplied from an external drug providing device will be aerosolized when transferred through the aerosolization device 71 of the nose part drug inlet 67.

In FIG. 2 the flow of drug through the inhalation arrangement 1 is illustrated. Drug is provided from external drug providing devices which in this embodiment are connected to both a pacifier part drug inlet 31 and a nose part drug inlet 67. However, in other embodiments of the invention drug may only be provided via the pacifier part drug inlet 31. Solid lines of the arrows illustrate drug provided via the pacifier part 3 and dashed lines of the arrows illustrate drug provided via the nose part 5. The nose part drug compartment 61 is connected to the pacifier part drug compartment 23 via the connection of the pacifier part 3 and the nose part 5 and drug can flow between the pacifier part drug compartment and the nose part drug compartment. Hereby enabling a versatile system where drug will be delivered through which ever orifice that is used for breathing, i.e. the nose or mouth or both. This minimizes the waste of drug.

In FIG. 3 an inhalation arrangement 101 according to another embodiment of the invention is shown in a perspective view. Most of the details are the same as for the embodiment as described in relation to FIGS. 1 and 2 and will not be described again. However, a pacifier part drug inlet 31′ and a gas inlet 41′ of the pacifier part 3′ are in this embodiment a bit different. The pacifier part drug inlet 31′ and the gas inlet 41′ are here combined in one connector which is configured for being connected by a tube 90′ to an external drug supplying device. Said external drug supplying device will in this embodiment supply both drug and gas via the tube 90′ into the pacifier part drug compartment 23 of the inhalation arrangement 101. The drug is in this embodiment already aerosolized. Hereby no aerosolization device is provided in the pacifier part 3′.

FIG. 4 is a perspective view of an inhalation arrangement 201 according to another embodiment of the invention. The pacifier part 3 is identical to the pacifier part 3 as illustrated in FIGS. 1, 2 and 7. However, the pacifier part could as well be the same as the pacifier part 3′ illustrated in FIG. 3. The nose part 5′ in this embodiment does not comprise a nose part drug inlet or any gas flow inlets. In this embodiment all drug is supplied via the pacifier part 3, i.e. from the external drug supplying device which is connected to the pacifier part drug inlet 31. Drug is transferred from the pacifier part drug compartment 23 to the nose part drug compartment 61 via the connection between the pacifier part and the nose part (in the same way as illustrated in FIG. 2). In this embodiment there is no connection of the nose part 5′ to an external gas flow, such as CPAP or HFNC and hereby the inhalation arrangement 201 according to this embodiment can be used also for example at home or other places without possibility for connection to medical equipment.

FIG. 5 is a perspective view of an inhalation arrangement 301 according to another embodiment of the invention. This is a similar embodiment as the one described in relation to FIG. 4 but with added connection capability of the nose part 5″ to an external gas providing device. Hereby the nose part 5 comprises gas flow inlets 69 connected to the nose part drug compartment 61 in the same way as described for the embodiment as shown in FIGS. 1 and 2. However, in this embodiment the nose part 5″ does not comprise a nose part drug inlet 67 as shown for the embodiment of FIGS. 1 and 2. All inhalation drug is instead provided via the pacifier part 3 as described for the embodiment of FIG. 4.

FIG. 6 is a perspective view of an inhalation arrangement 401 according to another embodiment of the invention. All the details are the same as for the embodiment described in relation to FIGS. 1 and 2 and will not be described again. However, the nose part 5′″ comprises a nose attachment part 63′ having a nose cap 64′ for transferring the drug into the nose of the user of the inhalation arrangement 401. The nose cap 64′ is configured for being attached to the nose of the user such that it covers the nostrils of the nose. Such a nose cap 64′ can be used instead of the nostril inert parts 64 in all the embodiments of the invention.

1. Inhalation arrangement wherein said inhalation arrangement comprises a pacifier part and a nose part which are configured to be connected to each other, said pacifier part comprising:
a nipple configured for being provided inside the mouth of a user of the inhalation arrangement, said nipple comprising a passageway, which is passing through the nipple, through which passageway a fluid can pass; and
a housing connected to the nipple, said housing comprising:
a pacifier part drug compartment (which is fluidly connected to the passageway of the nipple;
a pacifier part drug inlet configured for being connected to an external drug providing device, said pacifier part drug inlet being connected to the pacifier part drug compartment and configured for transferring a drug provided from a connected external drug providing device into the pacifier part drug compartment which is acting as a reservoir for the drug;
a gas inlet allowing air or other gas to enter into the pacifier part drug compartment; and
a nose part connector configured to be connected to a pacifier part connector of the nose part,
said nose part comprising:
a nose part drug compartment;
a nose attachment part connected to the nose part drug compartment and configured for transferring drug from the nose part drug compartment into a nose of a user of the inhalation arrangement; and
the pacifier part connector which is configured to be connected to the nose part connector of the pacifier part and hereby fluidly connecting the nose part drug compartment and the pacifier part drug compartment.

2. Inhalation arrangement according to claim 1, wherein the nose part further comprises a nose part drug inlet configured for being connected to an external drug providing device, said nose part drug inlet being connected to the nose part drug compartment and configured for transferring a drug provided from a connected external drug providing device into the nose part drug compartment.

3. Inhalation arrangement according to claim 1, wherein said nose part further comprises at least one gas flow inlet connected to the nose part drug compartment and configured for transferring gas from a connected external gas providing device into the nose part drug compartment.

4. Inhalation arrangement according to claim 1, wherein said pacifier part drug inlet comprises an aerosolization device through which said drug is transferred into the pacifier part drug compartment.

5. Inhalation arrangement according to claim 1, wherein the aerosolization device comprises a fine pore membrane, whereby a drug provided from a connected external drug providing device will be aerosolized when transferred through the fine pore membrane.

6. Inhalation arrangement according to claim 1, wherein the pacifier part drug inlet is configured for connection to a source of aerosolized drug

7. Inhalation arrangement according to claim 1, wherein the nose part connector is a closed valve when no nose part is connected to the pacifier part and is an open valve when a nose part is connected to the pacifier part.

8. Inhalation arrangement according to claim 1, wherein the nose attachment part comprises nostril inert parts or a nose cap.

9. Inhalation arrangement according to claim 1, wherein the pacifier part further comprises a shield, wherein the nipple is protruding from a first surface of the shield and the housing is provided on a second surface of the shield, which second surface is opposite to the first surface.

10. Inhalation arrangement according to claim 9, wherein the shield further comprises hooks for allowing securing the inhalation arrangement to a head of a user.
